(19) United States
(12) Patent Application Publication
(10) Pub. No.: US 2026/0124045 A1
(43) Pub. Date: May 7, 2026

(54) STACKABLE TIBIAL TRIAL INSERT ASSEMBLIES

(71) Applicant: NovoSource, LLC, Morgantown, WV (US)

(72) Inventors: Douglas Sherman Hawkins, Morgantown, WV (US); Jacob Scott Matheny, Westover, WV (US); Trevor Allen Schoonover, Masontown, WV (US)

(73) Assignee: NovoSource, LLC, Morgantown, WV (US)

(21) Appl. No.: 19/382,454

(22) Filed: Nov. 7, 2025

Related U.S. Application Data
(60) Provisional application No. 63/717,601, filed on Nov. 7, 2024.

(51) Int. Cl. A61F 2/46; A61F 2/30; A61F 2/38

(52) U.S. Cl. CPC A61F 2/4684; A61F 2/3094; A61F 2/389; A61F 2002/30553

(57) ABSTRACT

A tibial trial insert assembly is disclosed. The tibial trial insert assembly includes a baseplate trial, an insert trial selectively coupled to the baseplate trial, the insert trial including an insert trial body having a first insert trial surface and a second insert trial surface positioned opposite the first insert trial surface, the second insert trial surface abutting the baseplate trial in a coupled configuration, and a trial shim selectively coupled to the insert trial, the trial shim including a trial shim body having a first trial shim surface and a second trial shim surface positioned opposite the first trial shim surface, the second trial shim surface abutting the first insert trial surface of the insert trial in the coupled configuration.

### CROSS-REFERENCE TO RELATED APPLICATION

The present application claims the priority benefit of U.S. Provisional Application No. 63/717,601, entitled “STACKABLE TIBIAL TRIAL INSERT ASSEMBLIES” and filed November 7, 2024, the entire contents of which are incorporated herein by reference.

### BACKGROUND

### FIELD

The subject matter described herein relates to joint replacement, and more specifically, to trial insert assemblies for use during a trialling phase of a joint replacement procedure and methods of using the same.

### Technical Background

In a knee replacement surgery, such as a total knee arthroplasty, for example, damaged bone and/or cartilage are removed and replaced with an implant that recreates at least a portion of the joint. In various instances, a top surface of the tibia, or shinbone, can be replaced or supplemented with a tibial tray and a tibial insert. Such a tibial insert is typically placed on a flat metal platform (e.g., the tibial tray) allowing for stability using a cushion of strong, durable materials (e.g., plastic) to provide an ability of joint articulation, for example. The tibial insert is sized to fit between a femoral component and a tibial component of a total knee prosthesis. As anatomies and/or extent of injuries vary among subjects, an optimal thickness of the tibial insert can also vary.

Trialling is a phase of the knee replacement surgery in which one or more tibial trial inserts are temporarily positioned, or otherwise implanted, in an effort to determine an optimal size and/or geometry for a particular subject prior to replacing the affected bone and/or cartilage with a more permanent tibial insert. Testing tibial trial inserts of various sizes and/or geometries allows a user to judge flexion and extension gaps, soft tissue balancing, stability, and/or motion of the affected knee to increase successful surgery outcomes. Adjusting a geometrical parameter, such as a thickness, of the tibial trial insert during the trialling phase typically involves detaching a first trial insert portion from a base and replacing the first trial insertion portion with a second trial insert portion having a different geometrical parameter. Such removal and attachment of tibial trial inserts continues until a desirable result is achieved. However, trialling by removal and attachment of such tibial trial inserts is challenging and time consuming due to limited size constraints imposed by surrounding anatomical structures.

### SUMMARY

According to at least one aspect of the present disclosure, a tibial trial insert assembly is disclosed. The tibial trial insert assembly includes a baseplate trial, an insert trial selectively coupled to the baseplate trial, the insert trial including an insert trial body having a first insert trial surface and a second insert trial surface positioned opposite the first insert trial surface, the second insert trial surface abutting the baseplate trial in a coupled configuration, and a trial shim selectively coupled to the insert trial, the trial shim including a trial shim body having a first trial shim surface and a second trial shim surface positioned opposite the first trial shim surface, the second trial shim surface abutting the first insert trial surface of the insert trial in the coupled configuration.

According to at least one aspect of the present disclosure, a tibial trial insert assembly is disclosed. A tibial trial insert assembly includes a baseplate trial; an insert trial selectively coupled to the baseplate trial, where the insert trial includes an insert trial body having a first insert trial surface and a second insert trial surface positioned opposite the first insert trial surface and is abutting the baseplate trial in coupled configuration, where the first insert trial surface includes a concave first portion, a convex second portion, and a concave third portion, the convex second portion extending between the concave first portion and the concave third portion; and a trial shim selectively coupled to the insert trial, the trial shim including a trial shim body having a first trial shim surface and a second trial shim surface positioned opposite the first trial shim surface and is abutting the first insert trial surface of the insert trial, where the second trial shim surface includes a convex first portion, a concave second portion, and a convex third portion, where the concave second portion extends between the convex first portion and the convex third portion.

According to at least one aspect of the present disclosure, a method of assembling a tibial trial insert assembly in a procedure is disclosed. The method of assembling a tibial trial insert assembly in a procedure, the method includes coupling an insert trial including a first insert trial surface and a second insert trial surface positioned opposite the first insert trial surface to a baseplate trial to abut the second insert trial surface with the baseplate trial; and coupling a trial shim to the insert trial while maintaining the coupling of the insert trial to the baseplate trial, the trial shim including a first trial shim and a second trial shim surface positioned opposite the first trial shim surface, where the second trial shim is abutting the first insert trial surface.

### BRIEF DESCRIPTION OF THE FIGURES

The following detailed description of specific embodiments of the present disclosure can be best understood when read in conjunction with the following drawings, where like structure is indicated with like reference numerals and in which:

FIG. 1A is a perspective view of an illustrative tibial trial insert assembly including an insert trial coupled to a baseplate trial in accordance with at least one aspect of the present disclosure;

FIG. 1B is an exploded view of the tibial trial insert assembly of FIG. 1A in accordance with at least one aspect of the present disclosure;

FIG. 1C is a side view of the tibial trial insert assembly of FIG. 1A in accordance with at least one aspect of the present disclosure;

FIG. 2A is a perspective view of the tibial trial insert assembly of FIG. 1A further including an illustrative trial shim coupled to the insert trial in accordance with at least one aspect of the present disclosure;

FIG. 2B is an exploded view of the tibial trial insert assembly of FIG. 2A in accordance with at least one aspect of the present disclosure;

FIG. 2C is a side view of the tibial trial insert assembly of FIG. 2A as the trial shim is being coupled to the insert trial in accordance with at least one aspect of the present disclosure;

FIG. 2D is a perspective view of the tibial trial insert assembly of FIG. 2A as the trial shim is being coupled to the insert trial in accordance with at least one aspect of the present disclosure;

FIG. 2E is a side view of the tibial trial insert assembly of FIG. 2A depicting a completed assembly as the trial shim is coupled to the insert trial in accordance with at least one aspect of the present disclosure;

FIG. 3 depicts an exploded side view of the tibial trial insert assembly of FIG. 1A further including a plurality of illustrative stackable trial insert shims in accordance with at least one aspect of the present disclosure;

FIG. 4 depicts an exploded perspective view of the tibial trial insert assembly of FIG. 1 further including a plurality of illustrative stackable trial insert shims in accordance with at least one aspect of the present disclosure;

FIG. 5 depicts an exploded front view of the tibial trial insert assembly of FIG. 1 further including a plurality of illustrative stackable trial insert shims in accordance with at least one aspect of the present disclosure; and

FIG. 6 depicts a flow diagram of an illustrative overview method of assembling a tibial trial insert assembly during a knee replacement surgery in accordance with at least one aspect of the present disclosure.

### DETAILED DESCRIPTION

Use of conventional joint replacement components such as tibial trial insert assemblies oftentimes result in a process whereby components of the assembly are disassembled inside a subject’s body in order to adjust a characteristic of the assembly, such as a thickness, thereof. That is, the characteristic of the tibial trial insert assembly is generally adjusted by detaching a first component having a first thickness from the assembly installed in the subject’s body and replacing the first component with a second component having a different thickness. Notably, such detachment and insertion of assembly components within the subject’s body is tedious, time consuming, and difficult. In addition, one or more trial shims can be inserted into a conventional tibial trial insert assembly between a baseplate trial and an insert trial, for example. Such placement of the one or more trial shims between the baseplate trial and the insert trial also results in a similar process as described above whereby one or more components of the assembly are disassembled and replaced within the subject’s body.

Due to the tedious, time consuming, and difficult nature of disassembling, replacing, and reassembling components of the conventional assembly to adjust a characteristic of the assembly, such processes can have a number of drawbacks. For example, longer procedures generally necessitate additional operating room time, staffing, medicine delivery, consumable use, and/or the like. For example, a subject may be placed under anesthesia for a longer period of time, which can lead to complications. In another example, internal body parts of the subject are left exposed and subject to potential infection for a longer period of time. In still another example, as the cost of materials, surgical suite usage, and staffing continues to increase, the additional time needed using the conventional processes results in increased expenses, an increased risk in component contamination, and the like.

Conventionally, total knee arthroplasty procedures are performed in an ambulatory surgery center (ASC). Generally, the ASCs have limited storage capacity for sterilization equipment and other instrumentation. Conventional tibial trial insert assemblies require a large inventory of full-thickness tibial insert trials, which oftentimes necessitate additional space for multiple instrument trays, an increased sterilization workload, and increased costs.

Generally, conventional tibial trial insert assemblies include one or more trial shims that are substantially planar or flat in design. Such designs of the one or more trial shims do not effectively replicate the range of motion present in a natural knee joint. More specifically, such do not accurately reproduce the same joint kinematics as present in a natural knee joint. In particular, conventional tibial trial insert assemblies increase overall thickness by installing the one or more trial shims beneath the insert trial. Installing the trial shims beneath the insert trial oftentimes can interfere with the structure of the insert assembly, such as the baseplate trial walls, retaining members, or curvature features. Additionally, conventional tibial trial insert assemblies are oftentimes limited to the overall thickness due to limitations on how high the trial shims may be stacked when installed beneath the insert trial (e.g., physical limitations of the trial shims, such as particular mating features, size, shape, etc.).

Furthermore, conventional tibial trial insert assemblies oftentimes utilize polymeric materials, such as nylon, to form the trial inserts. Since conventional assemblies install the trial shims beneath the insert trial, the shims are tapped or impacted into position. The polymeric materials do not tolerate repetitive impaction forces and oftentimes repeated insertion and removal may lead to cracking, deformation, or breakage of the trial shims.

Accordingly, a need exists for a tibial trial insert assembly that remedies the deficiencies of the conventional tibial trial insert assemblies noted herein. More particularly, a need exists for tibial trial insert assemblies that include an insert trial and one or more trial shims with a first surface that has surface features that may mimic a typical joint. Such an insert trial and one or more trial shims with a curved surface effectively addresses issues with limited range of motion and inability to accurately replicate natural knee joint kinematics.

Additionally, a need exists for tibial trial insert assemblies where the one or more trial shims are configured to be stacked on top of the insert trial as opposed to being located between the baseplate trial and the insert trial to provide for better adjustment capabilities and reduce stress placed on the trial shims. Furthermore, a need exists for tibial trial insert assemblies where the trial insert does not need to be repeatedly removed from the baseplate trial. The capability to stack one or more trial shims on the insert trial effectively addresses issues with sanitation, sterilization, and efficiency as noted herein.

Embodiments of the stackable tibial trial insert assembly as described herein are configured for use during total knee arthroplasty procedures. The stackable tibial trial insert assembly includes an insert trial and one or more trial shims with a curved surface, which fulfill the previously described need by increasing the possible range of motion to substantially replicate a natural knee joint. Specifically, the femur bone is curved where it meets the patella and tibia in a natural knee joint. The curved design of the present insert trial and one or more trial shims may allow the tibial trial assembly to better mimic the design of a natural knee joint. Said another way, the curved surface of the insert trial and one or more shims become an extension of the femur bone to promote substantially similar ranges of motion to that of a natural knee joint. Specifically, the curved surface may provide positive axial alignment and rotational stability.

Additionally, embodiments of the tibial trial insert assembly as described herein include the one or more trial shims configured to be stackable on the insert trial to fulfill the previously described need by being more efficient for installation and providing long-term health of the knee joint and by aiding in better sanitation and sterilization standards.

In one instance, the present embodiments result in more efficient installation and long-term health of the knee joint because the one or more trial shims being configured to stack on top of the insert trial assists in the structural integrity of the femur bone. As previously mentioned, the end of the femur bone that meets the patella and tibia is curved in a natural knee joint. The present embodiment allows the trial shims to follow the natural structure (e.g., a trial shim with a congruent structure to the end of the femur bone) of the femur bone and provides the support directly on the femur bone and opposed to between two trial plates as present in conventional tibial trial insert assemblies. Following the natural structure of the bone further aids the femur bone retaining its natural integrity over a long-term. Additionally, the present embodiments allow for the stacking of non-congruent trial inserts that facilitate fixation using one or more anterior tabs or other fixation methods.

In a second instance, the present embodiments are more efficient for installation and long-term health of the knee joints because the one or more trial shims being configured to stack on top of the insert trial provides easier access for implanting the trial shims and more efficient alignment and fixation properties with the bone. Specifically, the present embodiments provide the practitioner with direct access to the joint to add the trial shims without uncoupling the baseplate trial and the insert trial as present in conventional tibial trial insert assemblies. Such direct access allows for the procedure to be less time-consuming, which allows for fewer staffing hours, less time needed for a surgical suite, fewer medicines being dispensed, less consumable use, and/or the like, leading to less expenses and/or less exposure time for both the subject and the practitioner. Additionally, the present embodiments may reduce the quantity of components needed to perform the procedure. Specifically, the present embodiments may be packed more compactly, sterilized more efficiently, and assist in the reduction of waste, which may be particularly advantageous in ASC environments.

Thus, such direct access allows the practitioner to make adjustments or alignments more efficiently and effectively. Said another way, the practitioner can add or remove one or more trial shims without decoupling the baseplate trial and the insert trial, thus resulting in an easier and/or more efficient procedure. Additionally, adding thickness above the insert trial may assist in preserving the stable engagement between insert trial and baseplate trial while also allowing for greater incremental adjustment of the overall thickness of the tibial trial insert assembly.

In a third instance, the present embodiments are more efficient for installation and long-term health of the knee joints because the one or more trial shims, in being configured to stack on the insert trial, aids in the reduction of stress during installation as compared to the conventional tibial trial insert assembly. Specifically, the present embodiments generally avoid a situation where the practitioner pries or forces apart the baseplate trial attached to the tibia and the insert trial attached to the femur to add or remove trial shims for adjustment purposes. Said another way, the present embodiments may assist in the prevention of damage or stretching of tissue (e.g., cartilage, ligaments, and/or the like) that surround the knee joint. Additionally, the process of prying or forcing the coupled baseplate trial and insert trial may prolong recovery in the subject. Thus, since excess stretching may occur in the conventional assemblies, the present assemblies are more beneficial for long-term health as it allows the surrounding muscle to retain its natural shape and structure.

Furthermore, the present embodiments generally avoid a situation where the practitioner pries, forces, or impacts the trial shims into place. Specifically, the present embodiments allow for the insert trial to remain seated on the baseplate trial while adjustments are made by adding or removing trial shims from above the insert trial. Said another way, the present embodiments reduce the mechanical stress played on the trial shims, which allows the shims to maintain consistent shape and structure and decreases any force imbued on the subject.

Embodiments of stackable tibial trial insert assemblies will now be described in more detail herein. The following will now describe these stackable tibial trial insert assemblies in more detail with reference to the drawings and where like numbers refer to like structures.

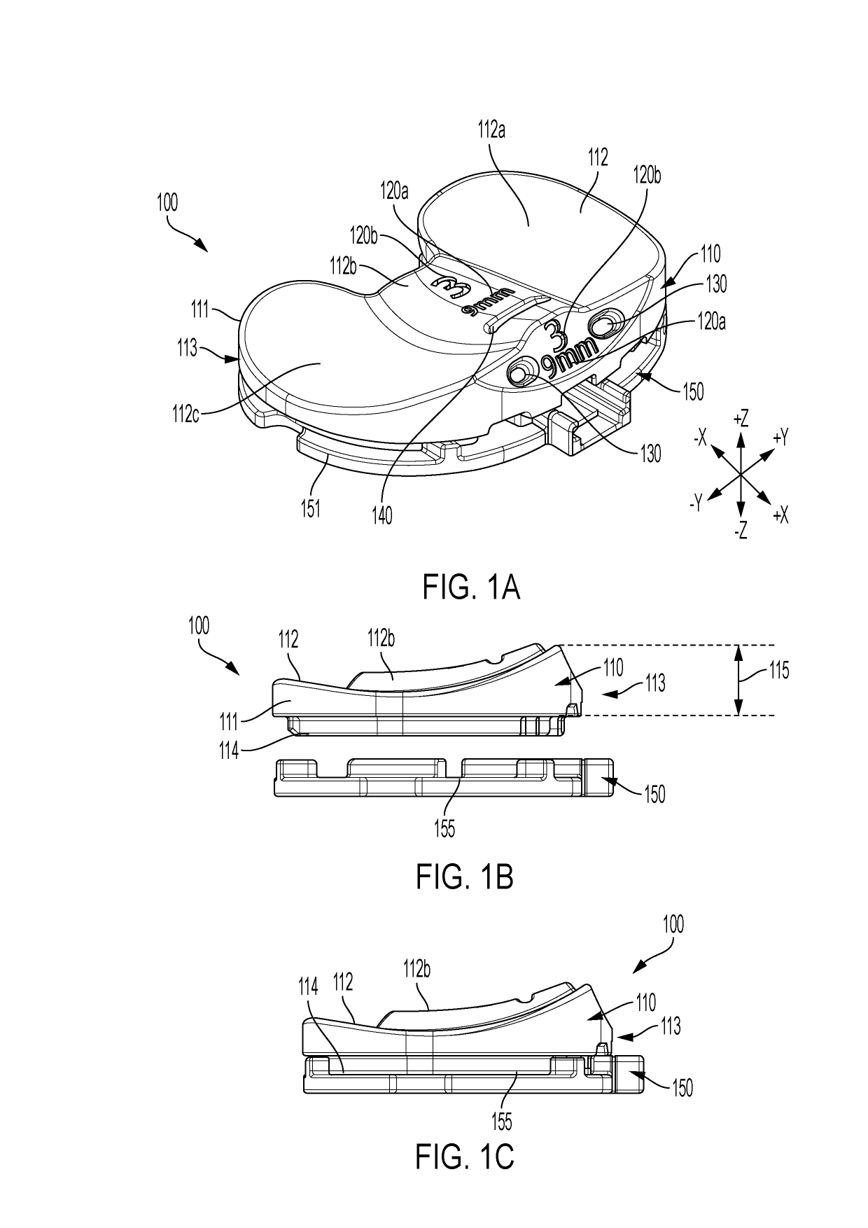

FIGS. 1A-1C depict a tibial trial insert assembly 100 including an insert trial 110 coupled, or otherwise connected, to a baseplate trial 150. The insert trial 110 includes a trial insert body 113 that further includes a first insert trial surface 112 and a second insert trial surface 114. The first insert trial surface 112 is opposite of the second insert trial surface 114. An insert trial thickness 115 is defined as a distance between the first insert trial surface 112 and the second insert trial surface 114. As depicted in FIGS. 1A-1C, the insert trial thickness 115 of the insert trial 110 is approximately nine (9) millimeters (mm). Typically insert trials are manufactured in a plurality of varying thicknesses, such as five to seven thicknesses, for example. More specifically, typical insert trials can be manufactured having thicknesses of about 9 mm, 11 mm, 13 mm, 15 mm, and 18 mm. An insert trial having a minimal thickness of 9 mm or 10 mm is most commonly utilized. As such, this disclosure bases its discussion on an insert trial 110 having a thickness of 9 mm or greater. Notably, any insert trial having any thickness can be utilized as part of the disclosed tibial trial insert assemblies.

Generally, the first insert trial surface 112 defines one or more concave regions. Stated another way, one or more regions of the first insert trial surface 112 are rounded inward (e.g., in a -Z direction of the coordinate axis), toward the second insert trial surface 114. More specifically, as shown in FIGS. 1A-1C, the first insert trial surface 112 includes a first portion 112a, a second portion 112b, and a third portion 112c. The first portion 112a and the third portion 112c are separated from one another by the second portion 112b, for example. Stated another way, the second portion 112b extends between and bridges the first portion 112a and the third portion 112c along the first insert trial surface 112. The first portion 112a and the third portion 112c are substantially mirror images, or reflections, of one another. The first insert trial surface 112 extending along the first portion 112a and the third portion 112c may be concave (e.g., defining a concave region). Stated another way, the first insert trial surface 112 is rounded inward (e.g., in a -Z direction of the coordinate axis) at points extending along the first portion 112a and the third portion 112c. In various instances, the first portion 112a and the third portion 112c may curve inward towards the second insert trial surface 114. In such instances, the concave nature of the first portion 112a and the third portion 112c may allow the first insert trial surface 112 to mimic the structure of the natural connection between a femur and a knee for more efficient or more natural kinematics of the knee of a subject. The concave nature of the first insert trial surface 112 is predominately shaped to configure to an end of the femur and may be distinguished from suitability for other joints.

The first insert trial surface 112 extending along the second portion 112b can be convex (e.g., defining a convex region). Stated another way, the first insert trial surface 112 is rounded outward (e.g., in a +Z direction of the coordinate axis), at one or more points extending along the second portion 112b. In various instances, the second portion 112b may curve outward away from the second insert trial surface 114.

In various instances, the one or more concave regions can be defined along the first portion 112a, the second portion 112b, and/or the third portion 112c, for example. That is, the first insert trial surface 112 may define a concave region at the first portion 112a, a concave region at the second portion 112b, and/or a concave region at the third portion 112c. In various instances, the second portion 112b is substantially planar or flat. In various instances, the first portion 112a may define a curvature, shape, or the like that is different from the third portion 112c. In various instances, the first insert trial surface 112 may define a uniform shape (e.g., defining a uniform concave region). Said a different way, the first portion 112a, the second portion 112b, and the third portion 112c may all define a similar or same curvature, shape, or the like.

The first insert trial surface 112 includes indicia 120 engraved, printed, and/or otherwise defined thereon in one or more locations so as to be readily visible by the user. More specifically, the indicia 120 can include alphanumeric characters, shapes, and/or colors, for example. The indicia 120 may be indicative of a characteristic of the insert trial 110, such as a geometrical characteristic, a dimension, a size, and/or a material of the insert trial 110, for example. As shown in FIG. 1A, a first indicia 120a is engraved, printed, and/or otherwise defined on the first insert trial surface 112 represents the thickness 115 of the insert trial 110. The thickness 115 of the insert trial 110 indicated by the first indicia 120a is nine (9) millimeters, for example. The first indicia 120a can be defined on one or more locations of the first insert trial surface 112. A second indicia 120b is engraved, printed, and/or otherwise defined on the first insert trial surface 112. As shown in FIG. 1A, the second indicia 120b represents a feature of the insert trial, such as a size of the insert trial, for example. In various instances, the size can correspond to a dimension of the insert trial such as a width, for example. The second indicia 120b can be defined on one or more locations of the first insert trial surface 112.

One or more cavities 130 are defined through at least a portion of the trial insert body 113 of the insert trial 110. In various instances, the one or more cavities 130 are through-holes. In other instances, the one or more cavities 130 do not extend through an entire thickness 115 of the insert trial 110. The one or more cavities 130 are sized to receive an attachment member of a trial shim 210, as discussed in greater detail herein. The one or more cavities 130 may be defined on the first insert trial surface 112. As shown in FIG. 1A, the one or more cavities 130 are defined on the first insert trial surface 112 adjacent a perimeter of the insert trial 110. However, the one or more cavities 130 may be defined on any suitable portion(s) of the first insert trial surface 112.

An aperture 140 is defined through at least a portion of the insert trial 110. In various instances, the aperture 140 is a through-hole. In other instances, the aperture 140 does not extend through an entire thickness 115 of the insert trial 110. As depicted in FIG. 1A, the aperture 140 is defined in the second portion 112b of the first insert trial surface 112. However, the aperture 140 can be defined on any suitable portion of the first insert trial surface 112. The aperture 140 is sized to receive a tool to facilitate removal of the insert trial 110 from the baseplate trial 150.

The second insert trial surface 114 is positioned abutting the baseplate trial 150 when the components of the tibial trial insert assembly 100 are in a coupled configuration. In general, a periphery 111 of the insert trial 110 is substantially similar to a periphery 151 of the baseplate trial 150. Stated another way, a length, a width, and/or a general border of the insert trial 110 substantially matches that of the baseplate trial 150 such that there are no significant portions of the baseplate trial 150 and/or the insert trial 110 extending beyond the other when the insert trial 110 is coupled to the baseplate trial 150.

The second insert trial surface 114 can include various attachment features that lock, or otherwise, engage with corresponding features of the baseplate trial 150. In various instances, the second insert trial surface 114 is substantially planar. In such instances, the second insert trial surface 114 is substantially flat, converse to the concave and/or convex profile of the first insert trial surface 112, for example. Having a substantially planar, or flat, second insert trial surface 114 facilitates a stable seating of the insert trial 110 against a correspondingly planar, or flat, mating surface 155 of the baseplate trial 150. In other instances, the second insert trial surface 114 is not substantially planar. Said another way, the second insert trial surface 114 may define a concave region, a convex region, or the like. In various instances, the second insert trial surface 114 may extend outward (e.g., in a -Z direction of the coordinate axis). In other instances, the second insert trial surface 114 may extend inward (e.g., in a -Z direction of the coordinate axis). Additionally, the second insert trial surface 114 may define a similar or same curvature, shape, or the like as the first insert trial surface 112. In such instances, a profile of the second insert trial surface 114 corresponds to a profile of the mating surface 155 of the baseplate trial 150 to facilitate a stable seating of the insert trial 110 therein.

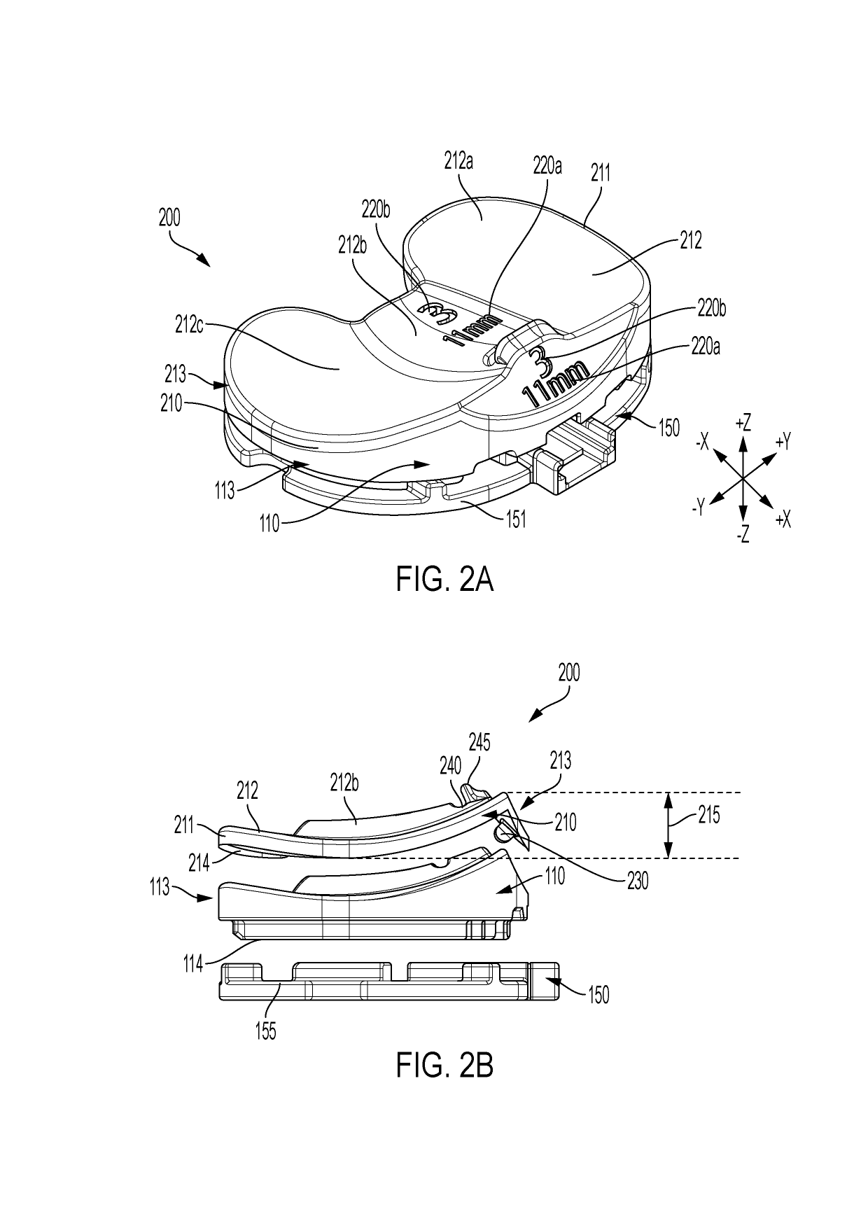

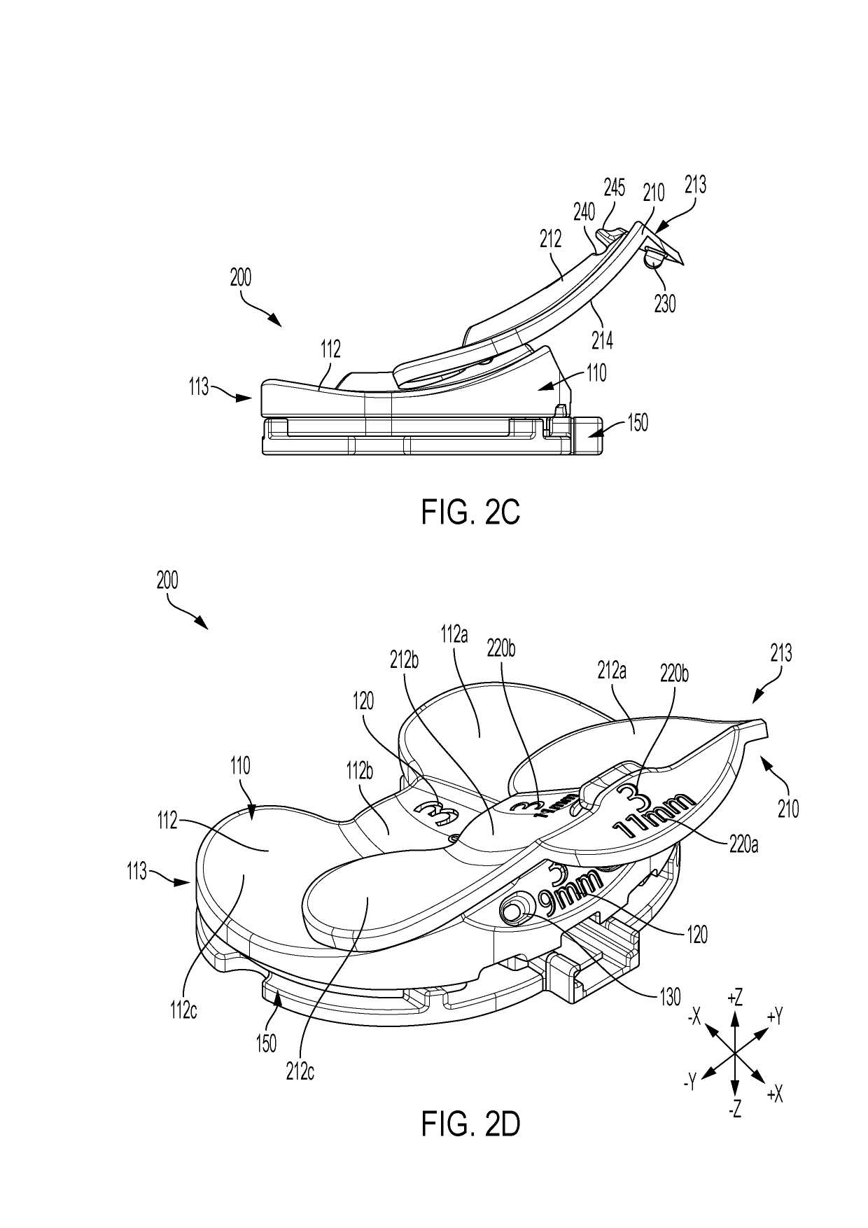

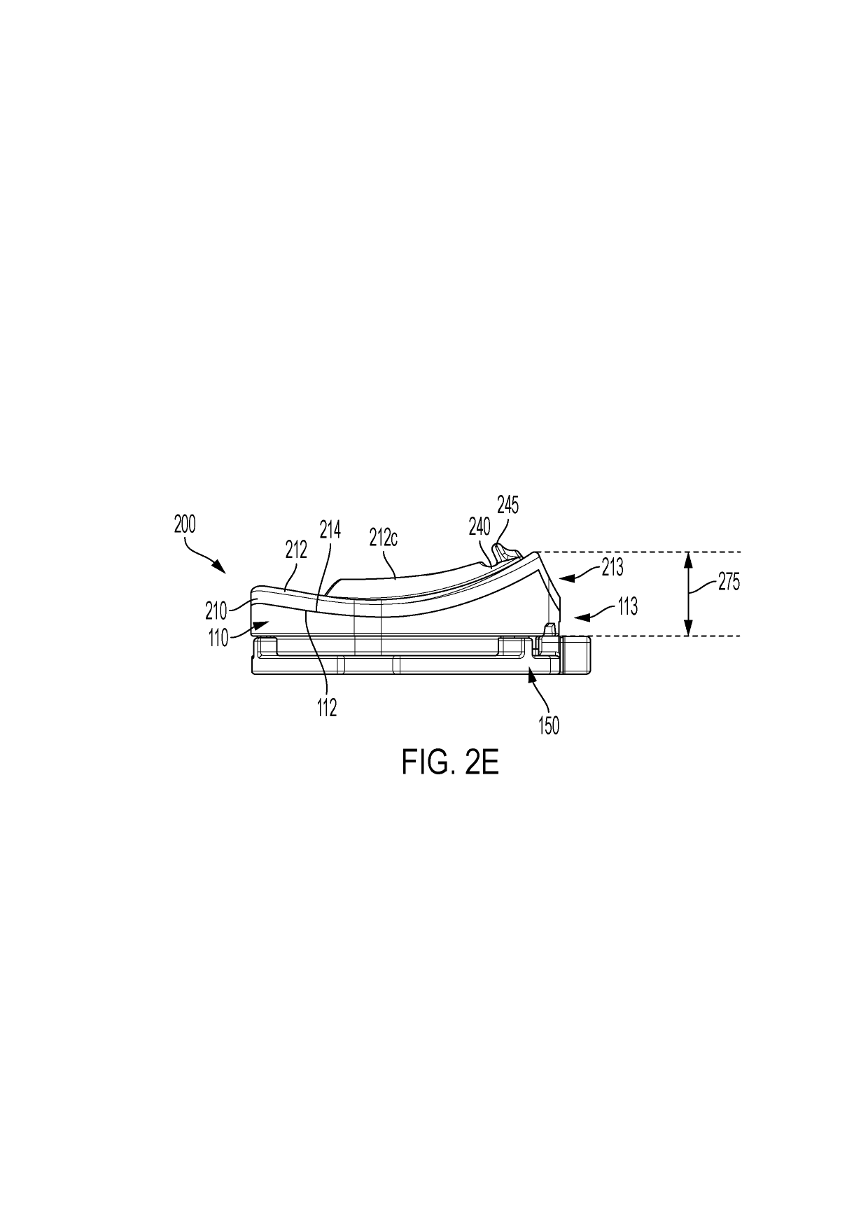

Referring now to FIGS. 2A-2E, a user may determine that a thickness 115 associated with the tibial trial insert assembly 100 is insufficient, or otherwise undesirable, for implantation into a particular subject. In instances where the user determines that additional thickness, or height, is desired or needed, the user may selectively couple, or otherwise attach, a trial shim 210 to the first insert trial surface 112 of the insert trial 110 to form a stack. As such, no existing components of the tibial trial insert assembly 100 are removed and/or replaced to form the new, thicker, tibial trial insert assembly 200.

The tibial trial insert assembly 200 depicted in FIGS. 2A-2E includes the insert trial 110 and the baseplate trial 150 of the tibial trial insert assembly 100. The tibial trial insert assembly 200 further includes one or more trial shims 210 having any desirable characteristic determined to improve the functionality of the tibial trial insert assembly 100 for a particular subject.

Similar in many respects to the insert trial 110, the trial shim 210 includes a trial shim body 213 that further includes a first trial shim surface 212 and a second trial shim surface 214. The first trial shim surface 212 is positioned opposite of the second trial shim surface 214. A trial shim thickness 215 is defined as a distance between the first trial shim surface 212 and the second trial shim surface 214. As depicted in FIGS. 2A-2E, the trial shim thickness 215 of the trial shim 210 is approximately two (2) millimeters (mm). However, any thickness of trial shim can be used. In instances where more than one trial shims 210 are coupled to the insert trial 110, the plurality of trial shims can have the same thickness as one another or the plurality of trial shims can have different thicknesses as one another. Stated another way, any thickness trial shim can be coupled to the tibial trial insert assembly 200 to achieve a desirable overall insert assembly thickness.

Generally, the first trial shim surface 212 includes one or more concave regions. Stated another way, one or more regions of the first trial shim surface 212 are rounded inward (e.g., in a -Z direction of the coordinate axis), toward the second trial shim surface 214. More specifically, as shown in FIGS. 2A-2E, the first trial shim surface 212 includes a first portion 212a, a second portion 212b, and a third portion 212c. The first portion 212a and the third portion 212c are separated from one another by the second portion 212b, for example. Stated another way, the second portion 212b extends between the first portion 212a and the third portion 212c along the first trial shim surface 212. The first portion 212a and the third portion 212c are substantially mirror images, or reflections, of one another. The first trial shim surface 212 extending along the first portion 212a and the third portion 212c may be concave (e.g., defining a concave region). Stated another way, the first trial shim surface 212 is rounded inward (e.g., in a -Z direction of the coordinate axis) at points extending along the first portion 212a and the third portion 212c. In various instances, the first portion 212a and the third portion 212c may curve inward towards the second trial shim surface 214. In such instances, the concave nature of the first portion 212a and the third portion 212c may allow the first trial shim surface 212 to mimic the structure of the natural connection between a femur and a knee for a more efficient or a more natural kinematics of the knee of a subject. The concave nature of the first trial shim surface 212 is predominately shaped to configure to an end of the femur and may be distinguished from suitability for other joints.

The first trial shim surface 212 extending along the second portion 212b can be convex (e.g., defining a convex region). Stated another way, the first trial shim surface 212 is rounded outward (e.g., in a +Z direction of the coordinate axis) at one or more points extending along the second portion 212b. In various instances, the second portion 212b may curve outward away from the second trial shim surface 214.

In various instances, the one or more concave regions can be defined along the first portion 212a, the second portion 212b, and/or the third portion 212c, for example. That is, the first trial shim surface 212 may define a concave region at the first portion 212a, a concave region at the second portion 212b, and a concave region at the third portion 212c. In various instances, the second portion 212b is substantially planar or flat. In various instances, the first portion 212a may define a curvature, shape, or the like that is different from the third portion 212c. In various instances, the first trial shim surface 212 may define a uniform shape (e.g., defining a uniform concave region). Said a different way, the first portion 212a, the second portion 212b, and the third portion 212c may all define a similar or same curvature, shape, or the like.

The first trial shim surface 212 includes indicia 220 engraved, printed, and/or otherwise defined thereon in one or more locations so as to be readily visible by the user. More specifically, the indicia 220 can include alphanumeric characters, shapes, and/or colors, for example. The indicia 220 may be indicative of a characteristic of the trial shim 210 and/or the tibial trial insert assembly 200, such as a geometrical characteristic, a dimension, a size, and/or a material of the trial shim 210 and/or the tibial trial insert assembly 200, for example. As shown in FIGS. 2A and 2E, a first indicia 220a is engraved, printed, and/or otherwise defined on the first trial shim surface 212 represents an overall thickness 275 associated with the tibial trial insert assembly 200. Such an overall thickness 275 is defined as a distance between a first trial shim surface 212 of the trial shim 210 and the second insert trial surface 114 of the insert trial 110.

The overall thickness 275 associated with the tibial trial insert assembly 200 indicated by the first indicia 220a is eleven (11) millimeters (mm), for example. Such an overall thickness 275 of 11 mm is a result of the coupling of a 2 mm thickness 215 trial shim 210 and a 9 mm thickness 115 insert trial 110 together. Notably, as shown in FIGS. 2A and 2D, the indicia 120 defined on the insert trial 110 is no longer visible to the user as the trial shim 210 is being coupled to the insert trial 110. As such, the user will not be confused as to the overall thickness 275 of the tibial trial insert assembly 200. The first indicia 220a can be defined on one or more locations of the first trial shim surface 212. A second indicia 220b is engraved, printed, and/or otherwise defined on the first trial shim surface 212. As shown in FIG. 2A, the second indicia 220b represents a feature of the insert trial, such as a size of the insert trial, for example. In various instances, the size can correspond to a dimension of the insert trial such as a width, for example. The second indicia 220b can be defined on one or more locations of the first trial shim surface 212.

In order to communicate an overall thickness 275, only particular trial shims 210 are compatible with a particular thickness of insert trial 110. More specifically, a first set of trial shims 210 are intended for use with an insert trial 110 having a thickness 115 of 9 mm. A second set of trial shims 210 are intended for use with an insert trial 110 having a thickness 115 of 11 mm, for example. Furthermore, the trial shims 210 within a particular set of trial shims are used in a particular order for the overall thickness 275 to be communicated accurately to the user. More specifically, in instances where a first trial shim having a thickness of 2 mm is coupled to an insert trial having a thickness of 11 mm, the first trial shim includes an indicia communicating an overall thickness of 13 mm, for example. Where a second trial shim 310 having a thickness of 2 mm is further coupled to the first trial shim 210, the second trial shim 310 includes an indicia indicative of an overall thickness of 15 mm, for example. In such instances, the second trial shim 310 cannot be coupled to the insert trial prior to the first trial shim or else the indicia of the second trial shim 310 may be indicative of an inaccurate overall thickness.

In various instances, the indicia 220 defined on the trial shim 210 is not indicative of a cumulative, or overall thickness 275 associated with the tibial trial insert assembly 200. Instead, the indicia 220 is indicative a thickness 215 of the trial shim 210. In such instances, the indicia 120 defined on the insert trial 110 remains visible to the user when the trial shim 210 is coupled to the insert trial 110 such that the user can readily deduce the overall thickness 275 associated with the tibial trial insert assembly 200.

An aperture 240 is defined through at least a portion of the trial shim body 213 of the trial shim 210. In various instances, the aperture 240 is a through-hole. In other instances, the aperture 240 does not extend through an entire thickness 215 of the trial shim 210. As depicted in FIGS. 2A, 2B, and 2E, the aperture 240 is defined in the second portion 212b of the first trial shim surface 212. However, the aperture 240 can be defined on any suitable portion of the first trial shim surface 212.

A projection 245 extends from the first trial shim surface 212 at a location adjacent the aperture 240. In instances where more than one trial shims 210 are coupled to the tibial trial insert assembly 200, the projection 245 of a first trial shim coupled to the insert trial 110 can extend through or into the aperture 240 of a second trial shim as the second trial shim is being coupled to the first trial shim. In various instances, the aperture 240 is sized to receive at least a portion of a tool to facilitate a removal of the second trial shim from an adjacent trial shim, such as insert trial 110 and/or the baseplate trial 150.

The second trial shim surface 214 of the trial shim 210 is positioned abutting the first insert trial surface 112 of the insert trial 110 when the components of the tibial trial insert assembly 200 are in coupled configuration. The second trial shim surface 214 substantially mimics the first insert trial surface 112 such that the trial shim 210 is stably seated on the insert trial 110. Stated another way, the second trial shim surface 214 is not flat. Instead, the second trial shim surface 214 includes convex portions intended to align with the concave portions (e.g., the first portion 112a and/or the third portion 112c) of the first insert trial surface 112, for example. Furthermore, the second trial shim surface 214 includes a concave portion intended to align with the convex portion (e.g., the second portion 112b) of the first insert trial surface 112. Such mimicking/corresponding geometries ensure a nested arrangement and facilitate a smooth and/or aligned insertion of the trial shim 210 to the tibial trial insert assembly 200 during the trialling phase.

As shown in FIGS. 2C and 2D, the trial shim 210 is configured to slidably engage the first insert trial surface 112 of the insert trial 110 as the trial shim 210 is being coupled to the insert trial 110. As the trial shim 210 is being slid relative to the insert trial 110, one or more attachment members 230 defined on the trial shim 210 engage the one or more cavities 130 defined in the insert trial 110. In various instances, the one or more attachment members 230 include a projection, a peg, a post, and/or the like. The interaction, or interface, between the one or more attachment members 230 and the one or more cavities 130 serves to maintain, or otherwise secure, the trial shim 210 to the insert trial 110 in a desired orientation. Furthermore, engagement between the one or more attachment members 230 and the one or more cavities 130 assists in ensuring to the user that the trial shim 210 is aligned with the insert trial 110, for example.

In general, a periphery 211 of the trial shim 210 is substantially similar to the periphery 111 of the insert trial 110 and/or the periphery 151 of the baseplate trial 150. Stated another way, a length, a width, and/or a general border of the trial shim 210 substantially matches that of the insert trial 110 and/or the baseplate trial 150 such that there are no significant portions of the insert trial 110 and/or the baseplate trial 150 extend beyond the periphery 211 of the trial shim 210 when the components of the tibial trial insert assembly 200 are coupled together.

In various instances, as shown in FIG. 2B, for example, the periphery 211 of the trial shim 210 includes a beveled, or otherwise rounded, edge along a portion thereof adjacent the second trial shim surface 214. The periphery 111 of the insert trial 110 includes a correspondingly beveled, or otherwise rounded edge, along a portion thereof adjacent the first insert trial surface 112. The beveled edge of the second trial shim surface 214 and the beveled edge of the first insert trial surface 112 align and engage one another as the trial shim 210 is coupled to the insert trial 110. Such an interaction provides an additional mechanism to maintain, or otherwise secure, the trial shim 210 to the insert trial 110 in the desired orientation.

In various instances, one or more components of the tibial trial insert assembly are comprised of nylon 12 or polyethylene. However, the one or more components of the tibial trial insert assemblies disclosed herein can be comprised of any suitable biocompatible material.

In various instances, one or more components of the tibial trial insert assembly can be sterilized. In various instances, one or more components of the tibial trial insert assembly are meant to be utilized once and subsequently disposed. In other instances, one or more components of the tibial trial insert assembly are intended to be reused more than once, for example.

It should be understood from the foregoing description that the trial shims may be stackable. That is, if a first trial shim (e.g., an 11 mm insert shim) is placed on a base insert and a thicker insert is desired or needed (e.g., 13 mm is desired or needed), a second trial shim (e.g., a 2 mm trial shim) could be placed (e.g., stacked) on the first trial shim instead of removing the first trial shim and placing a different trial shim in its place. Further, if additional thickness is still desired or needed, a third or subsequent trial shim could be placed on the first trial shim and the second trial shim to provide the additional thickness, and the process can be reiterated until the desired or needed thickness has been achieved. This stackable configuration allows for any number of trail shims to be added (e.g., 1 … n insert shims) without departing from the scope of the present application. Additionally, it should be understood that, in various instances, the thickness of the first trial insert may differ from the second trial shim, which the second trial shim may differ from any subsequent trial shim (e.g., third trial shim or fourth trial shim). Said another way, in those instances, the first trial shim is a desired thickness (e.g., a 2 mm trial shim) and the second trial shim is a different desired thickness (e.g., a 4 mm trial shim). In those aspects, the user can achieve a thicker desired thickness for the tibial trial insert assembly 100 without needing to place or stack a plurality of trial shims upon one another.

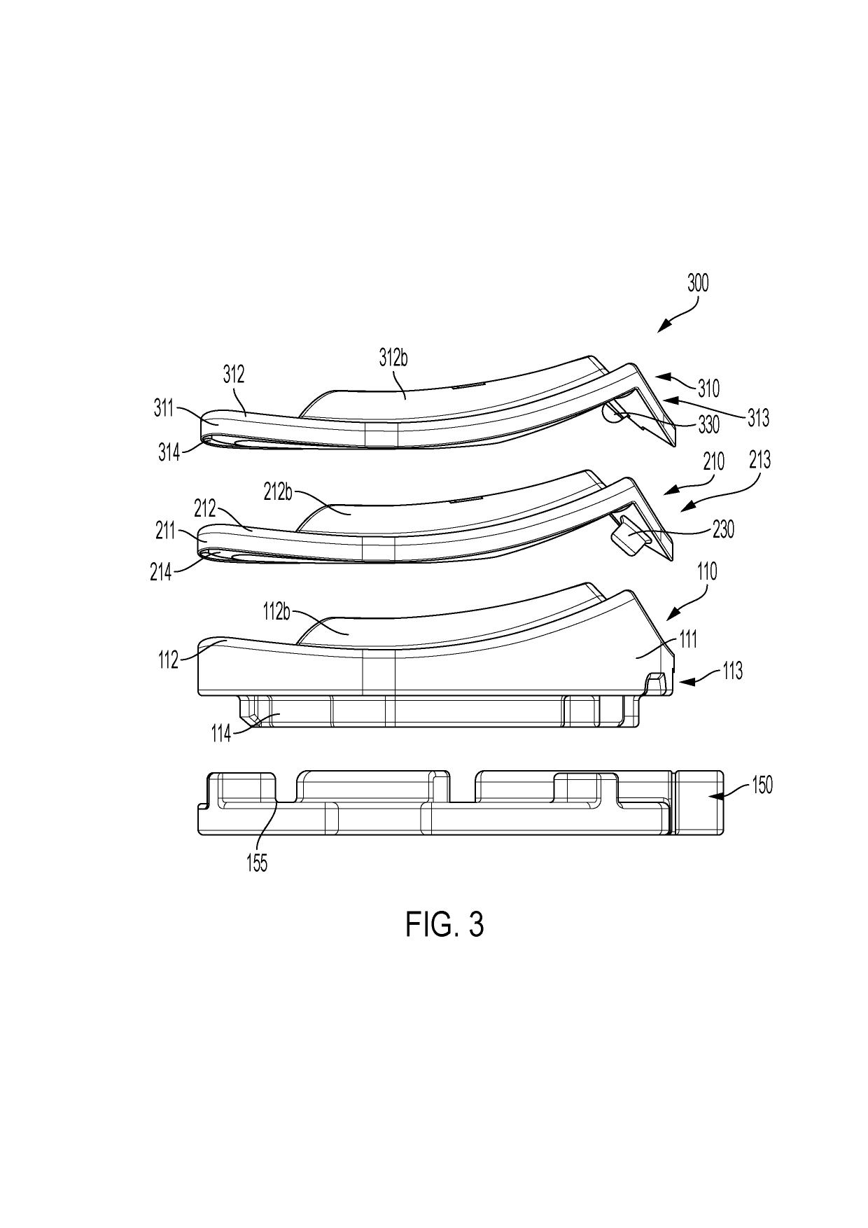

It should be understood that, in various instances, the plurality of trial shims may be coupled together away from a surgical site. Said another way, the plurality of trial shims may be selectively coupled, then the coupled plurality of trial shims can be selectively coupled to the insert trial 110 at the same time. In other instances, the plurality of the trial shims may be coupled to the insert trial 110 one at a time. Said another way, the second trial shim 310 may not be coupled to the first trial shim 210 until after the first trial shim 210 is selectively coupled to the insert trial 110. FIGS. 3-5 depict illustrative examples of stacking according to various aspects.

FIG. 3 depicts a tibial trial insert assembly 300 in an exploded side view. More specifically, FIG. 3 depicts the tibial trial insert assembly 100 and the tibial trial insert assembly 200 with an additional trial shim, herein referred to as a second trial shim 310. The tibial trial insert assembly 300 includes a baseplate trial 150, an insert trial 110, a trial shim 210, and a second trial shim 310. As noted hereinabove, the baseplate trial 150 includes a mating surface 155 and the insert trial 110 includes a first insert trial surface 112 and a second insert trial surface 114. The mating surface 155 is engageable with the second insert trial surface 114. Said another way, the baseplate trial 150 is configured to be coupled to the insert trial 110 by engaging the mating surface 155 to the second insert trial surface 114.

The trial shim 210 includes a first trial shim surface 212 and a second trial shim surface 214. The second trial shim surface 214 is slidably engageable with the first insert trial surface 112. Said another way, the insert trial 110 is configured to couple to the trial shim by engaging the first insert trial surface 112 with the second trial shim surface 214 through sliding engagement. The trial shim 210 further includes one or more attachment members 230 to secure engagement between the insert trial 110 and the trial shim 210.

In various aspects, the second trial shim 310 is substantially similar to the structure of the trial shim 210. Said another way, the second trial shim 310 includes a second trial shim body 313 that further includes a third trial shim surface 312, a fourth trial shim surface 314, and one or more attachment members 330. The fourth trial shim surface 314 is slidably engageable with the first trial shim surface 212 of the trial shim 210. Described differently, the trial shim 210 is configured to engage to the second trial shim by engaging the first trial shim surface 212 with the fourth trial shim surface 314. It should be appreciated that the tibial trial insert assembly 300 may include additional trial shims, such as a third trial shim, a fourth trial shim, etc., until the desired total thickness of the tibial trial insert assembly 300 is achieved.

In other aspects, the structure of the second trial shim 310 may remain substantially similar to the structure of the trial shim 210, but the thickness of the second trial shim 310 may be different or distinct from the thickness of the trial shim 210. Said another way, the trial shim 210 may have a desired thickness (e.g., a 2 mm trial shim) and the second trial shim 310 may have a different or distinct desired thickness (e.g., a 4 mm trial shim). In those aspects, the fourth trial shim surface 314 of the second trial shim 310 is slidably engageable with the first insert trial surface 112 of the insert trial 110. Said another way, the second trial shim 310 is only engageable with the insert trial 110 when the trial shim 210 has not previously been engaged with the insert trial 110. In those aspects, the trial shims 210, 310 are not stackable or engageable and a desired thickness is determined by a user selecting different trial shims 210, 310.

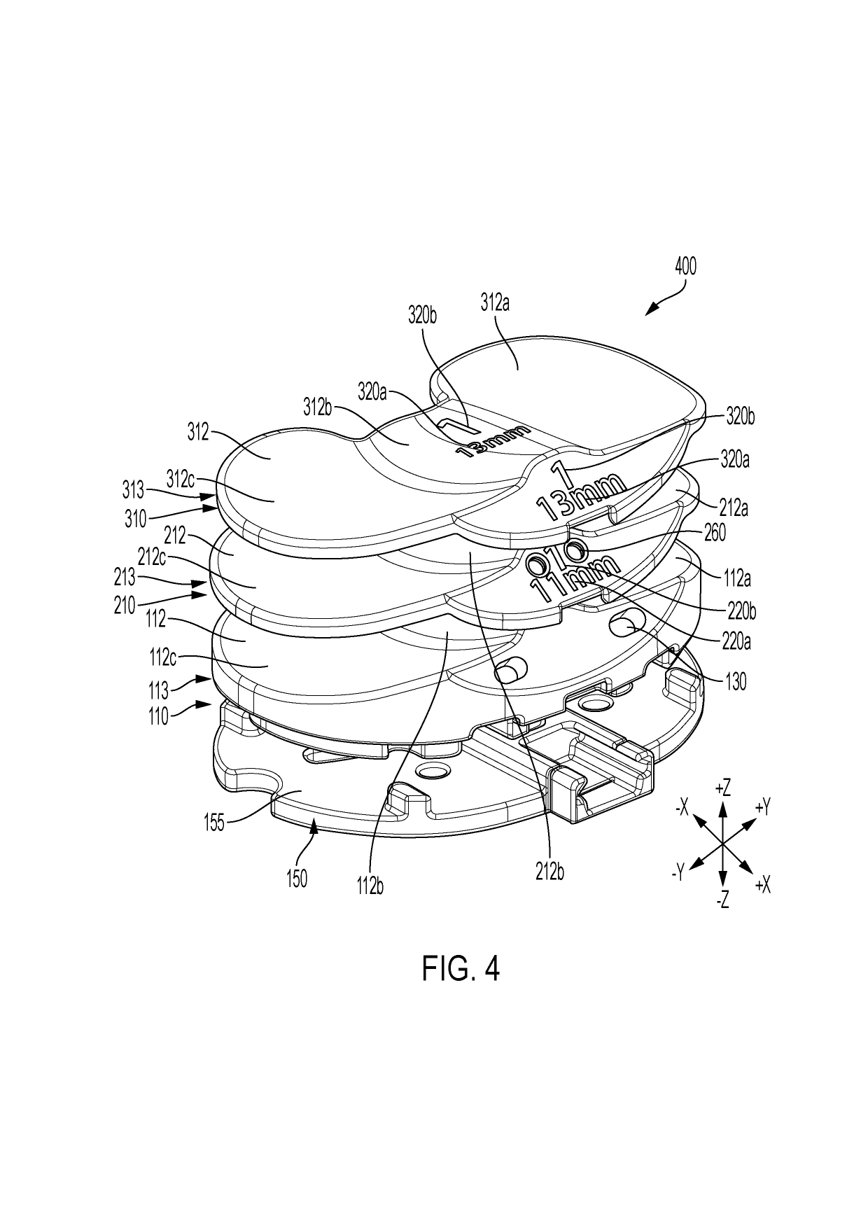

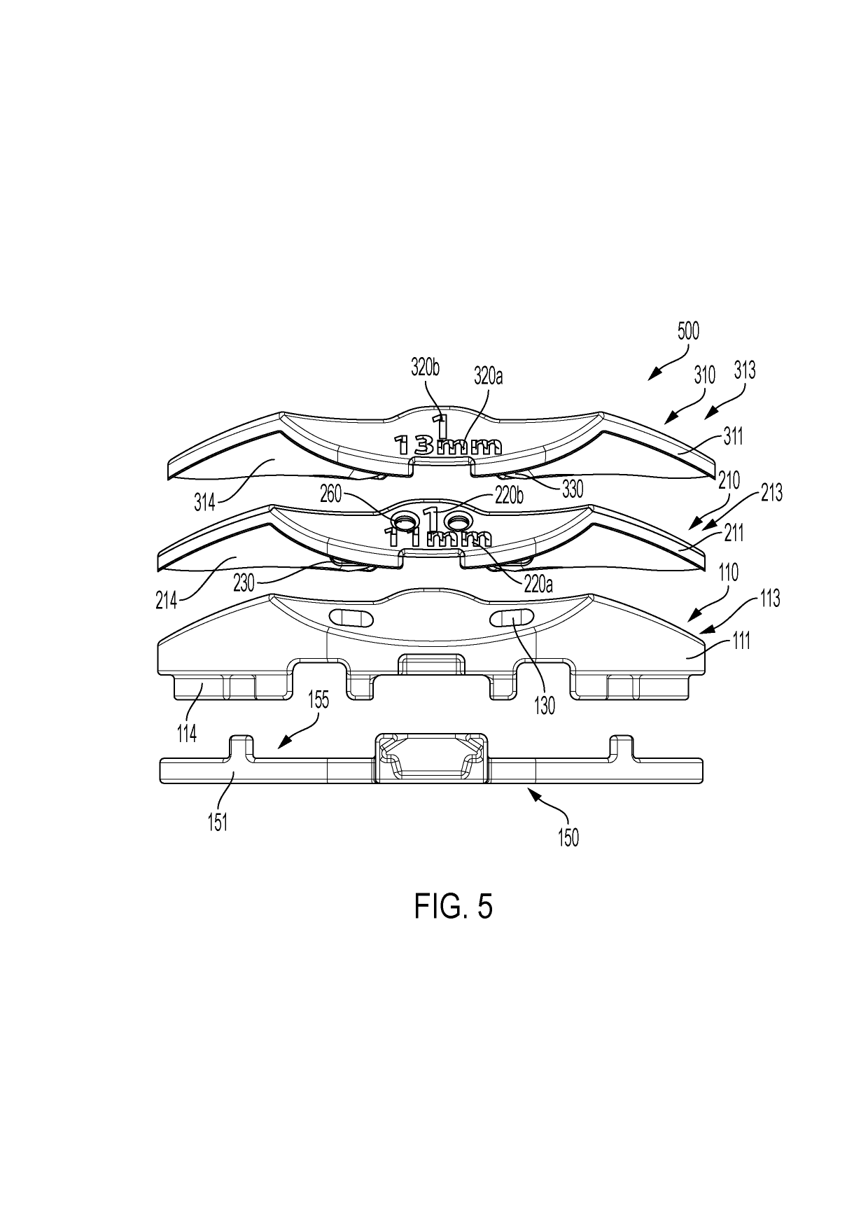

Referring now to FIG. 4 and FIG. 5. FIG. 4 depicts a tibial trial insert assembly 400 in an exploded perspective view. More specifically, FIG. 4 depicts the tibial trial insert assembly 100 and the tibial trial insert assembly 200 with an additional trial shim, herein referred to as a second trial shim 310. Additionally, FIG. 5 depicts a tibial trial insert assembly 500 in an exploded front view. More specifically, FIG. 5 depicts the tibial trial insert assembly 100 and the tibial trial insert assembly 200 with an additional trial shim, herein referred to as a second trial shim 310. The tibial trial insert assembly 400, 500 includes a baseplate trial 150, an insert trial 110, a trial shim 210, and a second trial shim 310.

The insert trial 110 includes a first insert trial surface 112 that is defined by a first portion 112a, a second portion 112b, and a third portion 112c. Said another way, the first portion 112a and the third portion 112c are separated from one another by the second portion 112b. In various aspects, the second portion 112b is substantially flat, planar, or the like. In other aspects, the second portion 112b is substantially convex (e.g., defining a convex region) or the like relative to the first portion 112a and the third portion 112c. In various aspects, the first portion 112a and the third portion 112c may be mirrored images, reflections, or the like. It should be appreciated that the first portion 112a and the third portion 112c may be distinct or substantially different in various embodiments. Additionally, the first portion 112a and the third portion 112c are substantially curved, concave, or the like. In such instances, the concave nature of the first portion 112a and the third portion 112c may allow the first insert trial surface 112 to mimic the structure of the natural connection between a femur and a knee for a more efficient and a more natural kinematics of the knee of a subject. The concave nature of the first insert trial surface 112 is predominately shaped to configure to an end of the femur and may be distinguished from suitability for other joints.

The insert trial 110 further includes a periphery 111 (FIG. 3) that extends around the body of the insert trial 110. The periphery 111 includes a correspondingly beveled, or otherwise rounded edge, along a portion thereof adjacent the first insert trial surface 112. The beveled edge of the periphery 111 may include one or more cavities 130 and indicia 120 (FIG. 2D). The indicia 120 is used to denote the overall thickness of the tibial trial insert assembly 400, 500. In various aspects, the peripheries 211, 311 (FIG. 3) of the trial shim 210 and second trial shim 310 may include indicia 220, 320.

The one or more cavities 130 of the insert trial 110 are engageable with the one or more attachment members 230 (FIG. 3) of the trial shim 210. Said another way, the insert trial 110 is configured to couple with the trial shim 210 by engaging the attachment members 230 with the cavities 130. Such an engagement is to provide an additional mechanism to maintain, secure, or the like the trial shim 210 to the insert trial 110 in the desired orientation. In the present aspect, a pair of cavities 130 is depicted. It should be appreciated that any number of cavities 130 may be disclosed including, but not limited to, one or more cavities 130. By way of non-limiting example, the number of cavities 130 may equal the number of attachment members 230.

The first trial shim surface 212 is substantially similar to the first insert trial surface 112. Said another way, the first trial shim surface 212 is defined by a first portion 212a, a second portion 212b, and a third portion 212c. In various aspects, the first portion 212a and the third portion 212c are separated from one another by the second portion 212b. Additionally, the second portion 212b is substantially flat, planar, or the like. In other aspects, the second portion 212b is substantially convex or the like relative to the first portion 212a and the third portion 212c. Furthermore, the first portion 212a and the third portion 212c may be mirrored images, reflections, or the like. It should be appreciated that the first portion 212a and the third portion 212c may be distinct or substantially different in various embodiments. Additionally, the first portion 212a and the third portion 212c are substantially curved, concave, or the like. In such instances, the concave nature of the first portion 212a and the third portion 212c may allow the first trial shim surface 212 to mimic the structure of the natural connection between a femur and a knee for a more efficient or a more natural kinematics of the knee of a subject. The concave nature of the first trial shim surface 212 is predominately shaped to configure to an end of the femur and may be distinguished from suitability for other joints.

The trial shim 210 further includes a periphery 211 (FIG. 3) that extends around the body of the trial shim 210. The periphery includes a correspondingly beveled, or otherwise rounded edge, along a portion thereof adjacent the first trial shim surface 212. The beveled edge of the periphery 211 may include one or more cavities 260 and indicia 220. The indicia 220 may include a first indicia 220a and a second indicia 220b. In various aspects, the first indicia 220a is engraved, printed, and/or otherwise defined on the first trial shim surface 212 and represents the overall thickness of the tibial trial insert assembly 400. In the present aspect, the first indicia 220a is depicted as 11-mm. It should be understood that the first indicia 220a represents the overall thickness of the tibial trial insert assembly 400 and not the thickness of the trial shim 210. The trial shim 210 may be any number of thicknesses including, but not limited to, a common trial shim thicknesses, such as 2-mm. In various aspects, the second indicia 220b is engraved, printed, and/or otherwise defined on the first trial shim surface 212 and represents a feature of the trial shim 210, such as a size of the trial shim 210.

The one or more cavities 260 of the trial shim 210 are engageable with the one or more attachment members 330 (FIG. 3) of the second trial shim 310. Said another way, the trial shim 210 is configured to couple with the second trial shim 310 by engaging the attachment members 330 with the cavities 260. Such an engagement is provided as an additional mechanism to maintain, secure, or the like the second trial shim 310 to the trial shim 210 in the desired orientation. In the present aspect, a pair of cavities 260 is depicted. It should be appreciated that any number of cavities 260 may be disclosed including, but not limited to, one or more cavities 260. By way of non-limiting example, the number of cavities 260 may equal the number of attachment members 330.

In various aspects, the second trial shim 310 is substantially similar to the structure of the trial shim 210. Said another way, the second trial shim 310 includes a periphery 311 (FIG. 3), a first indicia 320a, and a second indicia 320b. As described hereinabove, the first indicia 320a represents the overall thickness of the tibial trial insert assembly 400 and the second indicia 320b represents a feature of the second trial shim 310, such as the size of the second trial shim 310. The first indicia 320a and the second indicia 320b may be defined on one or more locations of the third trial shim surface 312. It should be appreciated that the tibial trial insert assembly 400, 500 may include additional trial shims, such as a third trial shim, a fourth trial shim, etc., until the desired total thickness of the tibial trial insert assembly 400 is achieved. In aspects with additional trial shims, the second trial shim 310 further includes one or more cavities that are configured to engage with one or more attachment members of any subsequent trial shims.

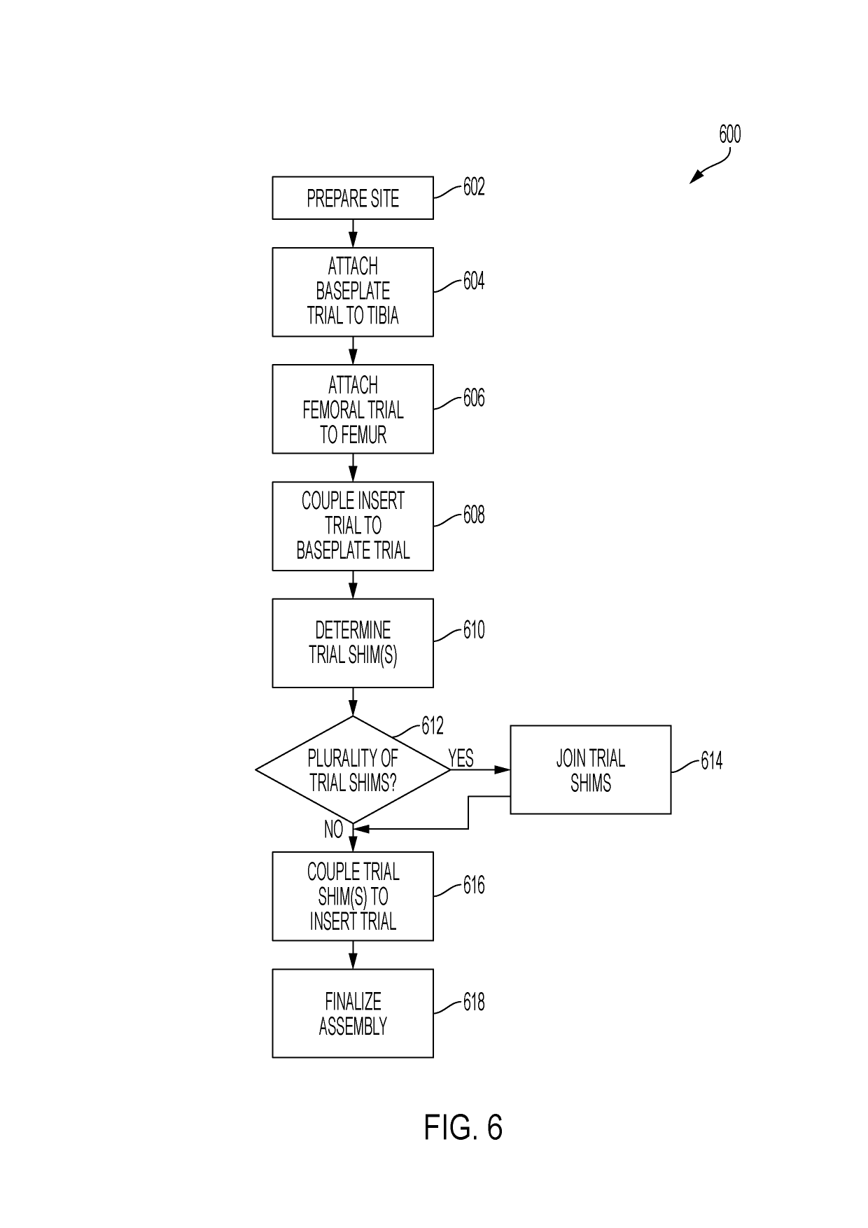

FIG. 6 depicts a flow diagram of an illustrative overview method of assembling a tibial trial insert assembly during knee replacement surgery 600. At block 602, a user prepares a surgical site. Said another way, preparing the surgical site may include, but is not limited to, the user cleaning a tibia and a femur of a subject, the user resurfacing the tibia and the femur of the subject, the user sterilizing a surgical area and/or surgical equipment, or the like.

At a block 604, a baseplate trial 150 is attached to the tibia of the subject. The baseplate trial 150 includes a mating surface 155 and a second surface opposite the mating surface 155. The second surface of the baseplate trial 150 is attachable to an end of the tibia of the subject.

At a block 606, a femoral trial is attached to the femur of the subject. The femoral trial includes a first surface and a second surface. The first surface of the femoral trial is attachable to an end of the femur of the subject. The second surface is configured to mimic the natural structure of the femur. The second surface of the femoral trial is further attachable to the final trial shim in coupled configuration with an insert trial 110.

At a block 608, the insert trial 110 is coupled to the baseplate trial 150. The insert trial 110 includes a first insert trial surface 112 and a second insert trial surface 114 that is position opposite to the first insert trial surface 112. The second insert trial surface 114 is positioned adjacent to the baseplate trial 150 and is engageable with the baseplate trial 150.

At a block 610, the user may determine the desired overall thickness 275 for the tibial trial insert assembly 100 to determine the number of trial shims 210 for the procedure. Said another way, at the block 612, the user may determine whether the procedure may be performed with one trial shim 210 or a plurality of trial shims 210.

In some aspects, the user may decide to float the tibia and the baseplate trial 150 for alignment purposes. Said another way, in those aspects, the baseplate trial 150 may not be attached to the tibia of the subject prior to performing the method at the block 606. In those aspects, the user may attach the baseplate trial 150 to the tibia at the block 604, couple the insert trial 110 to the baseplate trial 150 at the block 608, and determine the desired overall thickness 275 for the tibial trial insert assembly 100 at the block 610 after the femoral trial has been attached to the femur of the subject at the block 606.

If the user determines that the procedure may be performed with one trial shim 210, the user may proceed by coupling the trial shim 210 to the insert trial 110, as depicted in a block 616. If the user determines that the procedure may be performed with the plurality of trial shims 210, the user may proceed with joining the trial shims 210 before coupling the trial shims 210 to the insert trial 110, as depicted in a block 614.

At the block 614, a second trial shim 310, is coupled to the trial shim 210. The second trial shim 310 includes a third trial shim surface 312 and a fourth trial shim surface 314 that is positioned opposite to the third trial shim surface 312. The fourth trial shim surface 314 of the second trial shim 310 is positioned adjacent to the first trial shim surface 212 of the trial shim 210 and is engageable with the first trial shim surface 212 of the trial shim 210. In various instances, the user may join the trial shims 210, 310 together outside the surgical site and then couple the combined trial shims 210, 310 to the insert trial 110 at the same time. In other instances, the trial shims 210, 310 can coupled to the insert trial 110 and subsequent trial shims 210, 310 one at a time.

The process of the block 614 may be repeated a plurality of times with additional trial shims, such as a third trial shim or a fourth trial shim, to achieve the desired thickness of the tibial trial insert assembly 100 without departing from the scope of the present disclosure.

At a block 616, the trial shim 210 is coupled to the insert trial 110. In one instance, this coupling is performed without detaching the insert trial 110 from the baseplate trial 150. Said another way, the trial shim 210 may only be coupled to the insert trial 110 after the insert trial 110 has been coupled to the baseplate trial 150. In another instance, the trial shim 210 may be coupled to the insert trial 110 prior to the insert trial 110 being coupled to the baseplate trial 150. In that instance, the combination of the trial shim 210 and the insert trial 110 are then coupled to the baseplate trial 150. The trial shim 210 includes a first trial shim surface 212 and a second trial shim surface 214 that is positioned opposite of the first trial shim surface 212. The second trial shim surface 214 is positioned adjacent to the first insert trial surface 112 and is engageable with the first insert trial surface 112.

In other aspects, at the block 616, the second trial shim 310 may be coupled to the insert trial 110 without coupling the second trial shim 310 to the trial shim 210 or coupling the trial shim 210 to the insert trial 110. In those aspects, the thickness 215 of the trial shim 210 may be different or distinct from the thickness of the second trial shim 310. The difference in thicknesses between the trial shim 210 and the second trial shim 310 may allow the user to achieve a thicker overall thickness 275 of the tibial trial insert assembly 100 without requiring the user to place or stack a plurality of trial shims 210, 310 upon one another.

Those aspects can further allow the user to couple the trial shim 210 of a certain thickness to the insert trial 110, then remove the trial shim 210 from the insert trial 110 if the desired thickness is not achieved. Once the trial shim 210 has been removed, the user may couple the second trial shim 310 of a differing thickness from trial shim 210 to the insert trial 110. This process may be repeated a plurality of time until the user has achieved a desired overall thickness 275 for the tibial trial insert assembly 100.

At block 618, the user finalizes the tibial trial insert assembly 100 to complete the procedure. Said another way, the user may perform final alignment, stability, and fit tests to determine whether all components of the tibial trial insert assembly 100 are in coupled configuration. The user finalizes the assembly by securing, fastening, or the like the insert trial 110 and the trial shims 210, 310 of the tibial trial insert assembly 100.

Further aspects are provided by the subject matter of the following clauses:

According to a first aspect A1, a tibial trial insert assembly includes a baseplate trial; an insert trial selectively coupled to the baseplate trial, where the insert trial includes an insert trial body having a first insert trial surface and a second insert trial surface positioned opposite the first trial insert surface, where the second insert trial surface is abutting the baseplate trial in a coupled configuration; and a trial shim selectively coupled to the insert trial, where the trial shim includes a trial shim body having a first trial shim surface and a second trial shim surface positioned opposite the first trial shim surface, where the second trial shim surface is abutting the first insert trial surface of the insert trial in the coupled configuration.

A second aspect A2 includes the tibial trial insert assembly of any previous aspect, where the first insert trial surface defines at least one concave region.

A third aspect A3 includes the tibial trial insert assembly of any previous aspect, where the first insert trial surface further defines a convex region.

A fourth aspect A4 includes the tibial trial insert assembly of any previous aspect, where the second trial shim surface defines at least one convex region aligned with the at least one concave region of the first insert trial surface in coupled configuration.

A fifth aspect A5 includes the tibial trial insert assembly of any previous aspect, where a profile of the second trial mimics a profile of the first insert trial surface.

A sixth aspect A6 includes the tibial trial insert assembly of any previous aspect, where one or more cavities are defined in the first insert trial surface of the insert trial.

A seventh aspect A7 includes the tibial trial insert assembly of any previous aspect, where the trial shim further includes a projection extending from the second trial shim surface and where at least a portion of the projection is received within the one or more cavities as the trial shim is coupled to the insert trial.

An eighth aspect A8 includes the tibial trial insert assembly of any previous aspect, where the first insert trial surface of the insert trial includes an indicia.

A ninth aspect A9 includes the tibial trial insert assembly of any previous aspect, where the indicia is indicative of a thickness of the insert trial.

A tenth aspect A10 includes the tibial trial insert assembly of any previous aspect, where a the indicia is a first indicia, and where the first trial shim surface of the trial shim includes a second indicia.

An eleventh aspect A11 includes the tibial trial insert assembly of any previous aspect, where the second indicia is indicative of an overall thickness defined as a sum of the thickness of the insert trial and a thickness of the trial shim.

A twelfth aspect A12 includes the tibial trial insert assembly of any previous aspect, where the trial shim is a first trial shim, the tibial insert assembly further including a second trial shim selectively coupled to the first trial shim, the second trial shim including a second trial shim boy having a third trial shim surface and a fourth trial shim surface positioned opposite the third trial shim surface, and where the first trial shim surface is abutting the first trial shim surface of the trial shim in the coupled configuration.

A thirteenth aspect A13 includes the tibial trial insert assembly of any previous aspect, where the second trial shim includes an aperture defined therein, where the first trial shim includes a projection extending from the first trial shim surface, and where the aperture is sized to receive at least a portion of the projection.

According to a fourteenth aspect A14, a tibial trial insert assembly includes a baseplate trial; an insert trial selectively coupled to the baseplate trial, where the insert trial includes an insert trial body having a first insert trial surface and a second insert trial surface positioned opposite the first insert trial surface and is abutting the baseplate trial in coupled configuration, where the first insert trial surface includes a concave first portion, a convex second portion, and a concave third portion, the convex second portion extending between the concave first portion and the concave third portion; and a trial shim selectively coupled to the insert trial, the trial shim including a trial shim body having a first trial shim surface and a second trial shim surface positioned opposite the first trial shim surface and is abutting the first insert trial surface of the insert trial, where the second trial shim surface includes a convex first portion, a concave second portion, and a convex third portion, where the concave second portion extends between the convex first portion and the convex third portion.

A fifteenth aspect A15 includes the tibial trial insert assembly of any previous aspect, where the concave first portion of the first insert trial surface is aligned with the convex first portion of the second trial shim surface when the trial shim is selectively coupled to the insert trial, where the convex second portion of the first insert trial surface is aligned with the concave second portion of the second trial shim surface when the trial shim is selectively coupled to the insert trial, and where the concave third portion of the first insert trial surface is aligned with the convex third portion o the second trial shim surface when the trial shim is selectively coupled to the insert trial.

A sixteenth aspect A16 includes the tibial trial insert assembly of any previous aspect, where the convex second portion of the first insert trial surface includes an indicia.

A seventeenth aspect A17 includes the tibial trial insert assembly of any previous aspect, where the first insert trial surface includes the indicia, and where the indicia remains visible to a user when the trial shim and the insert trial are in coupled configuration.

An eighteenth aspect A18 includes the tibial trial insert assembly of any previous aspect, where the trial shim is couplable to the insert trial while maintaining a coupling between the insert trial and the baseplate trial.

According to a nineteenth aspect A19, a method of assembling a tibial trial insert assembly in a procedure, the method includes coupling an insert trial including a first insert trial surface and a second insert trial surface positioned opposite the first insert trial surface to a baseplate trial to abut the second insert trial surface with the baseplate trial; determining a number of one or more trial shims, where the one or more trial shims represent an overall thickness; coupling a first trial shim to the insert trial while maintaining the coupling of the insert trial to the baseplate trial, the first trial shim including a first trial shim and a second trial shim surface positioned opposite the first trial shim surface, where the second trial shim is abutting the first insert trial surface; and coupling a second trial shim to the first trial shim while maintaining the coupling of the first trial shim to the insert trial and the insert trial to the baseplate, the second trial shim comprising a third trial shim surface and a fourth trial shim surface positioned opposite the third trial shim surface, the fourth trial shim surface abutting the first trial shim surface.

A twentieth aspect A20 includes the method of assembling a tibial trial insert assembly in a procedure of any previous aspect, where the method includes decoupling the second trial shim from the first trial shim; decoupling the first trail shim from the insert trial; and coupling the second trial shim to the insert trial while maintaining the coupling of the insert trial to the baseplate, where the fourth trial shim surface of the second trial shim is abutting the first insert trial surface.

While various embodiments of the present disclosure are described with respect to tibial trial insert assemblies, such assembly components are envisioned for use with any type of implant, or prosthetic, trial insert assembly.

It will now be understood that the disclosure provides various solutions for optimizing a trialling process of a joint replacement procedure. The various solutions allow for a user to readily alter a characteristic of a trial insert assembly by adding one or more components to the trial insert assembly without having to disassemble the already-present components of the trial insert assembly. Additionally, the various solutions described herein provide various interfaces between the components of the trial insert assembly that facilitate insertion and/or alignment of the components thereof.

It is noted that the terms "substantially" and "about" may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

It is noted that the terms “selectively coupled” and “coupled configuration” may be utilized herein to represent the interaction between different components of the present disclosure. It should be understood that the term “selectively coupled” may mean that a connection may be specific between certain components while excluding the connection with other components based on various embodiments. Additionally, it should be understood that the term “coupled configuration” may mean that the components are unified by a connection becoming interconnected and interdependent to one another.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A tibial trial insert assembly, comprising:
a baseplate trial;
an insert trial selectively coupled to the baseplate trial, the insert trial comprising an insert trial body having a first insert trial surface and a second insert trial surface positioned opposite the first insert trial surface, the second insert trial surface abutting the baseplate trial in a coupled configuration; and
a trial shim selectively coupled to the insert trial, the trial shim comprising a trial shim body having a first trial shim surface and a second trial shim surface positioned opposite the first trial shim surface, the second trial shim surface abutting the first insert trial surface of the insert trial in the coupled configuration.

2. The tibial trial insert assembly of claim 1, wherein the first insert trial surface defines at least one concave region.

3. The tibial trial insert assembly of claim 2, wherein the first insert trial surface further defines a convex region.

4. The tibial trial insert assembly of claim 2, wherein the second trial shim surface defines at least one convex region aligned with the at least one concave region of the first insert trial surface in the coupled configuration.

5. The tibial trial insert assembly of claim 1, wherein a profile of the second trial shim surface mimics a profile of the first insert trial surface.

6. The tibial trial insert assembly of claim 1, wherein one or more cavities are defined in the first insert trial surface of the insert trial.

7. The tibial trial insert assembly of claim 6, wherein the trial shim further comprises a projection extending from the second trial shim surface, and wherein at least a portion of the projection is received within the one or more cavity as the trial shim is coupled to the insert trial.

8. The tibial trial insert assembly of claim 1, wherein the first insert trial surface of the insert trial includes an indicia.

9. The tibial trial insert assembly of claim 8, wherein the indicia is indicative of a thickness of the insert trial.

10. The tibial trial insert assembly of claim 9, wherein the indicia is a first indicia, and wherein the first trial shim surface of the trial shim includes a second indicia.

11. The tibial trial insert assembly of claim 10, wherein the second indicia is indicative of an overall thickness defined as a sum of the thickness of the insert trial and a thickness of the trial shim.

12. The tibial trial insert assembly of claim 1, wherein the trial shim is a first trial shim, the tibial trial insert assembly further comprising:
a second trial shim selectively coupled to the first trial shim, the second trial shim comprising a second trial shim body having a third trial shim surface and a fourth trial shim surface positioned opposite the third trial shim surface, the fourth trial shim surface abutting the first trial shim surface of the trial shim in the coupled configuration.

13. The tibial trial insert assembly of claim 12, wherein the second trial shim comprises an aperture defined therein, wherein the first trial shim comprises a projection extending from the first trial shim surface, and wherein the aperture is sized to receive at least a portion of the projection.

14. A tibial trial insert assembly, comprising:
a baseplate trial;
an insert trial selectively coupled to the baseplate trial, the insert trial comprising an insert trial body having a first insert trial surface and a second insert trial surface positioned opposite the first insert trial surface and abutting the baseplate trial in a coupled configuration, the first insert trial surface comprising:
a concave first portion;
a convex second portion; and
a concave third portion, the convex second portion extending between the concave first portion and the concave third portion; and
a trial shim selectively coupled to the insert trial, the trial shim comprising a trial shim body having a first trial shim surface and a second trial shim surface positioned opposite the first trial shim surface and abutting the first insert trial surface of the insert trial, the second trial shim surface comprising:
a convex first portion;
a concave second portion; and
a convex third portion, wherein the concave second portion extends between the convex first portion and the convex third portion.

15. The tibial trial insert assembly of claim 14, wherein the concave first portion of the first insert trial surface is aligned with the convex first portion of the second trial shim surface when the trial shim is selectively coupled to the insert trial, wherein the convex second portion of the first insert trial surface is aligned with the concave second portion of the second trial shim surface when the trial shim is selectively coupled to the insert trial, and wherein the concave third portion of the first insert trial surface is aligned with the convex third portion of the second trial shim surface when the trial shim is selectively coupled to the insert trial.

16. The tibial trial insert assembly of claim 14, wherein the convex second portion of the first insert trial surface includes an indicia.

17. The tibial trial insert assembly of claim 14, wherein the first insert trial surface includes the indicia and the indicia remains visible to a user when the trial shim and the insert trial are in coupled configuration.

18. The tibial trial insert assembly of claim 14, wherein the trial shim is couplable to the insert trial while maintaining a coupling between the insert trial and the baseplate trial.

19. A method of assembling a tibial trial insert assembly in a procedure, the method comprising:
coupling an insert trial comprising a first insert trial surface and a second insert trial surface positioned opposite the first insert trial surface to a baseplate trial to abut the second insert trial surface with the baseplate trial;
determining a number of one or more trial shims, wherein the one or more trial shims represent an overall thickness;
coupling a first trial shim to the insert trial while maintaining the coupling of the insert trial to the baseplate trial, the first trial shim comprising a first trial shim surface and a second trial shim surface positioned opposite the first trial shim surface, the second trial shim abutting the first insert trial surface; and
coupling a second trial shim to the first trial shim while maintaining the coupling of the first trial shim to the insert trial and the insert trial to the baseplate, the second trial shim comprising a third trial shim surface and a fourth trial shim surface positioned opposite the third trial shim surface, the fourth trial shim surface abutting the first trial shim surface.

20. The method of claim 19, further comprising:
decoupling the second trial shim from the first trial shim;
decoupling the first trial shim from the insert trial;
coupling the second trial shim to the insert trial while maintaining the coupling of the insert trial to the baseplate, wherein the fourth trial shim surface of the second trial shim is abutting the first insert trial surface.
